SEQUENCE LISTING (4 sequences)

SEQ ID NO: 1 - DNA, 35 bp, Artificial Sequence
  primer
ataggatccacctggccagcgagttcaagaagaag

SEQ ID NO: 2 - DNA, 30 bp, Artificial Sequence
  primer
tatctcgagttatttgggcttcatctccac

SEQ ID NO: 3 - DNA, 39 bp, Artificial Sequence
  primer
gctcgagcctaagttccaccatggccacgagtcaggaag

SEQ ID NO: 4 - DNA, 37 bp, Artificial Sequence
  primer
tcttatctagaagcttttatttgggcttcatctccac

(19) United States
(12) Patent Application Publication
(10) Pub. No.: US 2016/0317613 A1
(43) Pub. Date: Nov. 3, 2016

(54) ULTRASOUND-ASSISTED GENE TRANSFER FOR TREATMENT OF XEROSTOMIA

(71) Applicant: Allegheny-Singer Research Institute, Pittsburgh, PA (US)

(72) Inventor: Michael J. Passineau, Pittsburgh, PA (US)

(21) Appl. No.: 15/144,683

(22) Filed: May 2, 2016

Related U.S. Application Data
(60) Provisional application No. 62/155,917, filed on May 1, 2015.

(51) Int. Cl. A61K 38/17; A61M 37/00; A61K 41/00; A61K 9/00; A61K 48/00

(52) U.S. Cl. CPC A61K 38/177; A61K 9/006; A61K 48/0075; A61K 41/0047; A61M 37/0092

(57) ABSTRACT

Methods and compositions useful for gene transfer into a salivary gland are provided. In certain aspects, methods and compositions useful in the treatment of hyposalivation and/or xerostomia are provided. In particular aspects, methods may include contacting a salivary gland of a subject with a composition containing microbubbles and a non-viral expression vector containing a nucleic acid sequence encoding a water channel, and applying ultrasound to the salivary gland.

### CROSS-REFERENCE TO RELATED APPLICATION

This patent document claims the benefit of U.S. Provisional Patent Application No. 62/155,917, filed May 1, 2015, titled “ULTRASOUND-ASSISTED GENE TRANSFER FOR TREATMENT OF XEROSTOMIA,” the disclosure of which is hereby incorporated by reference herein in its entirety.

### STATEMENT REGARDING FEDERALLY SPONSORED RESEARCH

This invention was made, at least in part, with government support under grant number DE022973 awarded by the National Institutes of Health. The government has certain rights in the invention.

### BACKGROUND

Cancers of the head and neck (HNC) comprise roughly 3% of all cancer cases in the United States, with an incidence estimated at 52,000 cases in the United States in 2013. Incidence of these cancers varies globally, according to the prevalence of risk factors in various populations. Throughout the developed world, external beam radiation remains a mainstay of therapy for most types and stages of HNC, either alone or in combination with surgery and/or chemotherapy. Coirradiation of the salivary glands during radiotherapy is common and results in severe and irreversible hyposalivation, which in turn leads to a constellation of oral morbidities including xerostomia and dental disease.

Treatment options for radiation-induced xerostomia are extremely limited and consist of exogenous rehydration and management of progressive damage to the oral mucosa and dentition. In light of these limited treatment options and the chronic and debilitating nature of this condition, gene therapy for radiation-induced xerostomia previously has been developed and successfully demonstrated in a Phase I clinical trial (Baum B J, et al. Proc Natl Acad Sci USA. 2012 Nov. 20; 109(47):19403-7; Baum B J, et al. Biochim Biophys Acta. 2006 August; 1758(8):1071-7. Epub 2005/12/22. eng.). This gene therapy strategy is targeted to the ductal cells of the salivary gland which, in contrast to the saliva-producing acinar cells, are resistant to ionizing radiation and survive radiotherapy largely intact. Using an Adenoviral vector to express Aquaporin 1 (AdAQP1) in parotid gland ductal cells, a treatment paradigm was developed that resulted in the transcellular flux of interstitial fluid across the ductal cell layer and into the intraductal labyrinth of the salivary gland, where the fluid can be expelled to produce palliative oral wetness.

This AdAQP1 clinical trial established the safety of AQP1 gene therapy, as well as demonstrating objective improvements in parotid salivary flow rates and subjective improvement in xerostomia in patients receiving the treatment in a dose-dependent manner. Despite this success, a Phase II trial of AdAQP1 is not planned, as the therapeutic effect is transient and this vector is not considered suitable for the readministration required to treat this chronic condition. Adenovirus elicits strong host immune response in humans, and this response is thought to be progressive with repeated exposure.

While hyposalivation and/or xerostomia are not life threatening or life limiting, the impact of these conditions on quality of life, nutrition, oral and digestive health is severe and can be devastating. When it is considered that patients suffering from radiation-induced xerostomia, as well as other causes of xerostomia such as Sjogren's syndrome and drug-related xerostomia, can live for decades with this condition, the development of a clinically practicable salivary gland gene therapy has the potential to mitigate much suffering.

### SUMMARY

In one aspect, various embodiments described in this document relate to a method for gene transfer into a salivary gland of a subject comprising contacting a salivary gland of the subject with a composition comprising microbubbles and a non-viral expression vector comprising a nucleic acid sequence encoding a water channel and applying ultrasound to the salivary gland.

In another aspect, various embodiments described in this document relate to a method for treating xerostomia and/or hyposalivation in a subject in need of treatment comprising contacting a salivary gland of the subject with a composition comprising microbubbles and a non-viral expression vector comprising a nucleic acid sequence encoding a water channel and applying ultrasound to the salivary gland.

In another aspect, various embodiments described in this document relate to a method of increasing salivary flow from an undamaged salivary gland in a subject comprising contacting a salivary gland of the subject with a composition comprising microbubbles and a non-viral expression vector comprising a nucleic acid sequence encoding a water channel and applying ultrasound to the contacted salivary gland, wherein salivary flow is increased from an undamaged contralateral salivary gland.

In another aspect, various embodiments described in this document relate to a method of treatment for xerostomia comprising:

A) providing to a salivary gland of a subject in need thereof a treatment comprising administering a composition comprising microbubbles and a non-viral expression vector comprising a nucleic acid sequence encoding a water channel, and applying ultrasound to the salivary gland;

B) assessing the level of xerostomia in the subject using a visual analog scale (VAS); and

C) if the assessment indicates that xerostomia remains present in the subject, providing further treatment to the salivary gland, wherein the treatment comprises administering a composition comprising microbubbles and a non-viral expression vector comprising a nucleic acid sequence encoding a water channel, and applying ultrasound to the salivary gland.

Optionally, the above steps A-C may be repeated until assessment by visual analog scale indicates that xerostomia has been relieved in the subject.

### BRIEF DESCRIPTION OF THE DRAWINGS

FIG. 1 illustrates, in graphical and table format, the experimental group assignments for 16 animal subjects and the treatments, sample collections, and experimental endpoint of each group.

FIGS. 2A-B illustrate a dosimetry study and radiation-induced hyposalivation.

FIGS. 3A-B illustrate ultrasound-assisted non-viral gene transfer to the parotid gland of a Yucatan minipig.

FIG. 4 illustrates AdhAQP1 gene therapy in a swine model of radiation-induced hyposalivation.

FIGS. 5A-B illustrate testing of pAQP1 channel function.

FIG. 6 illustrates pAQP1 gene therapy using UAGT in irradiated pig model.

FIG. 7 shows representative histology of parotid glands 1 week following Adenoviral gene transfer and UAGT.

FIG. 8 shows proteomic profiling of pooled saliva.

FIG. 9 illustrates the effect of multiple UAGT treatments on reporter gene expression in the submandibular gland in mice.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art.

The disclosure of the following patent document is incorporated herein by reference: U.S. Patent Application Publication No. 2011/0124716, published May 26, 2011, titled “Ultrasound-Assisted Gene Transfer to Salivary Glands.”

A Phase I human clinical trial utilizing Adenoviral gene transfer of Aquaporin-1 (AQP1) to a single salivary gland of individuals suffering from radiation-induced xerostomia has previously been reported. Unfortunately, the limitations of the Adenoviral vector system utilized in this trial preclude its advancement in further clinical trials. The present inventor has thus undertaken to demonstrate the therapeutic potential of ultrasound-assisted non-viral gene transfer (UAGT) as an alternative means of delivering AQP 1 gene therapy to the salivary gland. In one aspect, this was accomplished by comparing UAGT head-to-head with the Adenoviral vector in a swine model. Swine irradiated unilaterally with a 10 Gy electron beam targeted at the parotid gland suffered from significant, sustained hyposalivation that was bilateral, despite irradiation being confined to the targeted gland. Unilateral AQP1 gene therapy with UAGT resulted in bilateral restoration of stimulated salivary flow at 48 hours and one week post-treatment (1.62+/−0.48 ml, 1.87+/−0.45 ml) to pre-injury levels (1.34+/−0.14 ml) in a manner comparable to Adenoviral delivery (2.32+/−0.6 ml, 1.33+/−0.97 ml). These results demonstrate that UAGT can replace the Adenoviral vector as a means of delivering AQP1 gene therapy in the irradiated swine model.

Further, UAGT may provide a suitable method for gene therapy in human patients. Successful application of gene therapy for hyposalivation and/or xerostomia may be dependent upon the clinical implementation of a gene therapy technique that evades host immune response and allows for periodic readministration throughout the lifetime of the individual.

In certain aspects, embodiments disclosed in this document relate to ultrasound- assisted gene transfer (UAGT) to the salivary gland, which may combine the use of a non-viral DNA vector and microbubbles with a low frequency acoustic field to create a “sonoporation” effect, allowing gene transfer to the cells of the salivary gland without the introduction of viral antigens. This method has been shown to successfully express transgenes within the salivary gland of rodents and has advantages over conventional viral vectors. In certain embodiments, this method may utilize safe and clinical-grade components, including, but not limited to: 1) clinical ultrasound, 2) perflutren/lipid microbubbles which have been approved for intravascular administration to humans, and 3) non-viral DNA vectors. While intracellular host immune response can still occur with non-viral DNA vectors, extracellular host response, either humoral or cell-mediated, is thought to be minimized in the absence of viral antigens, provided that the transgene itself is native to the host.

The present inventor has developed and characterized a swine model of radiation-induced xerostomia, effected UAGT to the parotid gland of swine subjects, and demonstrated the therapeutic efficacy and duration of AQP1 gene therapy in the swine by directly comparing UAGT and Adenoviral gene transfer. As the swine is the penultimate preclinical model of radiation-induced xerostomia, efforts were designed to mimic those of the AdAQP1 preclinical development effort, and to provide a rationale for the translation of UAGT/AQP1 to treatment for human patients suffering from radiation-induced xerostomia. These efforts provide an alternative to viral-based gene therapy, and, by obviating host immune response, establish a gene therapy protocol that may be periodically re-administered throughout the lifetime of the patient.

The present inventor has demonstrated herein that UAGT can provide effective gene therapy for radiation-induced xerostomia in the penultimate preclinical model, the irradiated miniature swine. A novel swine model is described herein, wherein irradiation of the salivary gland is accomplished with an electron beam, obviating the possibility of radiation damage to the contralateral gland. Based upon this model, a novel observation is described reporting that unilateral damage to one parotid gland impairs the function of the contralateral gland.

The observation that the functions of the left and right parotid glands are functionally coupled is likely of great clinical importance. Two previous studies in a similar irradiated pig model noted possible reduction in salivary flow from the uninjured gland, but as these studies utilized photons, it was assumed that the contralateral effects were due to some spillover and/or scatter of radiation from the targeted side. In the present results, that possibility has been excluded, demonstrating that the reduction in flow on the contralateral side is related in some way to damage to the targeted gland.

The present inventor has demonstrated the efficacy of non-viral UAGT to the parotid glands of swine. The present technique delivers therapeutic benefit equivalent to the clinically-validated Adenoviral gene therapy approach, but obviates the need to expose the patient to the systemic toxicity and local inflammation associated with the Adenoviral vector. This host immune response to viral vectors has historically presented the greatest challenge to the mainstreaming of gene therapy, and in the case of gene therapy for radiation-induced xerostomia, this limitation makes Adenovirus unsuitable for advancement to a Phase II trial. Significant morphological and functional damage to the salivary glands following Adenoviral gene transfer has been noted in animal studies and herein is reported data confirming these safety considerations. Damage to the salivary gland resulting from Adenoviral gene transfer may similarly occur in humans. Alternative strategies, such as Adeno-associated virus (AAV) delivery of AQP1 may be evaluated, but would still necessarily involve re-exposing the patient to a viral vector every few years, risking progressive anti-vector host response.

The proteome of the fluid produced by the treated salivary gland as a result of the gene therapy intervention described herein (UAGT/pAQP1) is qualitatively very similar to natural saliva but lacks certain proteins normally supplied by the parotid acinar. The clinical significance of this observation is complex. On the one hand, the fluid produced is believed to be palliative, and is expected to relieve subjective xerostomia in patients, as it is reported to have done in the AdAQP1 clinical trial. On the other hand, the absence or alternate isoforms of major salivary proteins such as Amylase, Lactoferrin and Lactoperoxidase (Table 2) makes it possible that the fluid produced may lack the full efficacy of natural saliva in preventing caries and preserving the overall health of the oral mucosa.

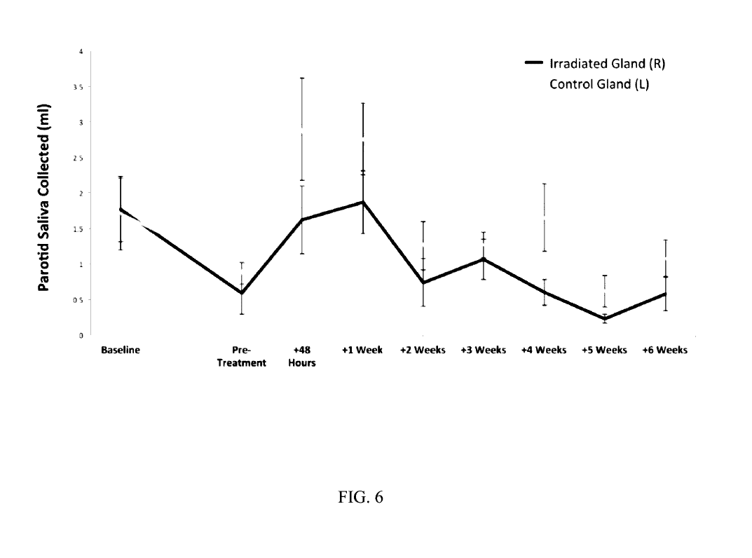

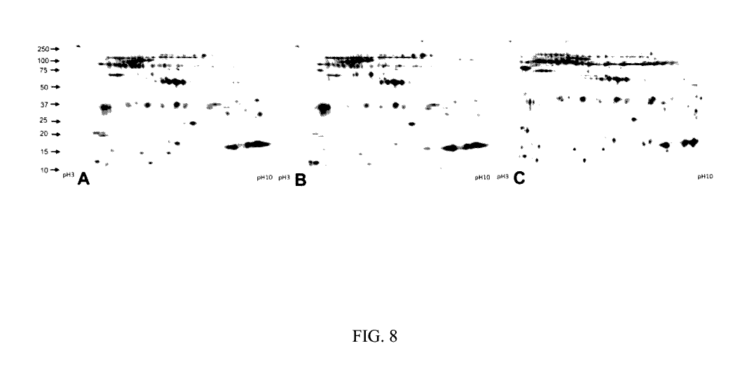

The ability of AQP1 gene therapy delivered to the irradiated parotid gland to restore and even augment the function of the contralateral, undamaged gland is noteworthy. Integrating area-under-the-curve in FIG. 6, it appears that the majority of the saliva produced as a result of the UAGT/AQP1 gene therapy intervention actually came from the undamaged, untreated control parotid gland. As shown in FIG. 8, the global similarity in the proteome of Baseline (8A) and saliva from the contralateral gland (Control) (8B), compared to UAGT/pAQP1 saliva from the treated gland (8C), is visually evident. This indicates that UAGT of one gland enables the production of saliva from the contralateral, untreated salivary gland that contains the complement of proteins found in normal saliva. This principle likely holds true in human patients, such that the benefit of UAGT to the maintenance of oral and dental health may be very significant.

The following conditions should be noted, and are imposed either due to logistical challenges with this large animal model or economy of animal use for experimental design. First, the 10 Gy radiation is a mild insult, and is delivered as a single dose, in contrast to the fractionated dosing used clinically. As described below, a 20 Gy electron beam causes severe radiation burns, and ongoing experiments suggest that lower doses of radiation, such as, for example, 15 Gy may be suitable. Second, immunohistochemical localization of pAQP1 expression was not shown in treated minipigs, although immunohistochemical distribution of a transgene in the salivary glands of mice following UAGT was previously shown.

Based upon the findings described herein, UAGT may be used to achieve expression of suitable gene products in human salivary glands. Further, UAGT of a human gene product may reduce hyposalivation and/or xerostomia in human patients. In embodiments, human gene products, such as human water channels, may be used. In embodiments, human aquaporins may be used. In embodiments, human aquaporin-1 (hAQP1) may be used.

Modeling using pAQP1 in pigs is likely to be a sound way of determining the duration of therapeutic effect following UAGT, absent concerns of host response against a foreign transgene (i.e., hAQP1), which could of themselves limit the duration of therapeutic effect.

The present inventor has demonstrated herein that UAGT can replace Adenovirus as the gene transfer technology to carry forward as a gene therapy for hyposalivation and/or xerostomia.

In embodiments described in this document, any suitable non-viral expression vector may be used. In embodiments, a plasmid vector may be used. Minimalist plasmid constructs, termed “minicircles” or “mini-intronic plasmids” delete all or most foreign backbone sequences. In embodiments, the present UAGT gene therapy strategy may be suitable for use with minicircles, which may further reduce unwanted effects. Additionally, in embodiments, UAGT may be suitable for the delivery of gene editing components (including, but not limited to, CRISPR/Cas) to a salivary gland as a means of permanent insertion of a suitable gene product, including, but not limited to, an aquaporin, into surviving ductal cells.

In embodiments, microbubbles may be bubbles smaller than one millimeter in diameter, but larger than one micrometer. They are filled with a gas, which, in embodiments, may include but is not limited to, air or a perfluorocarbon; this enables the microbubbles to oscillate and vibrate when a sonic energy field is applied. Microbubbles are encapsulated with a solid shell, which may be made from a material that, in embodiments, may include but is not limited to, a lipid or a protein.

In embodiments, suitable microbubble compositions may include those having a lipid bilayer surrounding a perfluoropropane gas core (e.g, DEFINITY, available from Lantheum Medical Imaging), and microbubbles having perfluoropropane gas encapsulated by a serum albumin shell (e.g., OPTISON, available from GE healthcare).

In embodiments, suitable water channel proteins include, but are not limited to, aquaporins. In an embodiment, the water channel is aquaporin-1. In embodiments, suitable water channel proteins include human aquaporins. In an embodiment, the water channel protein is human aquaporin-1.

Certain embodiments provide a method of treating a subject. As used herein, the term “subject” is used to mean an animal, preferably a mammal, including a human. The terms “patient” and “subject” may be used interchangeably.

In certain embodiments, methods are provided for gene transfer into a salivary gland of a subject comprising contacting a salivary gland of the subject with a composition comprising microbubbles and a non-viral expression vector comprising a nucleic acid sequence encoding a water channel and applying ultrasound to the salivary gland.

In embodiments, the composition comprises the non-viral vector at a concentration of about 0.01 μg/μl to about 1 μg/μl, about 0.01 μg/μl to about 0.2 μg/μl, about 0.01 μg/μl to about 0.1 μg/μl, or about 0.1 μg/μl to about 1 μg/μl. In embodiments, the composition comprises the non-viral vector in an amount of about 1.76×10⁹ copies of the vector/μl to about 1.76×10¹¹ copies of the vector/μl, about 1.76×10⁹ copies of the vector/μl to about 1.76×10¹⁰ copies of the vector/μl, or about 1.76×10¹⁰ copies of the vector/μl to about 1.76×10¹¹ copies of the vector/μl.

In embodiments, the salivary gland is a parotid gland. In embodiments, the salivary gland is a submandibular gland.

In embodiments, suitable water channel proteins include, but are not limited to, aquaporins. In an embodiment, the water channel is aquaporin-1. In embodiments, suitable water channel proteins include human aquaporins. In an embodiment, the water channel protein is human aquaporin-1.

In embodiments, the ultrasound may be applied at a frequency of about 0.5 MHz to about 2.5 MHz, or about 1 MHz to about 2 MHz.

In embodiments, the saliva volume is increased in the contacted salivary gland. In embodiments, saliva volume is increased in an untreated contralateral salivary gland. In an embodiment, saliva volume is increased in an untreated contralateral parotid gland.

In embodiments, a method is provided for treating xerostomia and/or hyposalivation in a subject in need of such treatment comprising contacting a salivary gland of the subject with a composition comprising microbubbles and a non-viral expression vector comprising a nucleic acid sequence encoding a water channel, and applying ultrasound to the salivary gland.

In embodiments, the composition comprises the non-viral vector at a concentration of about 0.01 μg/μl to about 1 μg/μl, 0.01 μg/μl to about 0.2 μg/μl, about 0.01 μg/μl to about 0.1 μg/μl, or about 0.1 μg/μl to about 1 μg/μl. In embodiments, the composition comprises the non-viral vector in an amount of about 1.76×10⁹ copies of the vector/μl to about 1.76×10¹¹ copies of the vector/W, about 1.76×10⁹ copies of the vector/μl to about 1.76×10¹⁰ copies of the vector/μl, or about 1.76×10¹⁰ copies of the vector/μl to about 1.76×10¹¹ copies of the vector/μl.

In embodiments, the salivary gland is a parotid gland. In embodiments, the salivary gland is a submandibular gland.

In embodiments, suitable water channel proteins include, but are not limited to, aquaporins. In an embodiment, the water channel is aquaporin-1. In embodiments, suitable water channel proteins include human aquaporins. In an embodiment, the water channel protein is human aquaporin-1.

In embodiments, the ultrasound may be applied at a frequency of about 0.5 MHz to about 2.5 MHz, or about 1 MHz to about 2 MHz.

In embodiments, saliva volume is increased in the contacted salivary gland. In embodiments, saliva volume is increased in an untreated contralateral salivary gland. In an embodiment, saliva volume is increased in an untreated contralateral parotid gland.

In embodiments, methods are provided for increasing salivary flow from an undamaged salivary gland in a subject comprising contacting a salivary gland of the subject with a composition comprising microbubbles and a non-viral expression vector comprising a nucleic acid sequence encoding a water channel, and applying ultrasound to the contacted salivary gland, wherein salivary flow is increased from an undamaged contralateral salivary gland.

In embodiments, the contacted gland is a parotid gland. In embodiments, salivary flow is increased from an undamaged contralateral parotid gland.

In embodiments, the composition comprises the non-viral vector at a concentration of about 0.01 μg/μl to about 1 μg/μl, 0.01 μg/μl to about 0.2 μg/μl, about 0.01 μg/μl to about 0.1 μg/μl, or about 0.1 μg/μl to about 1 μg/μl. In embodiments, the composition comprises the non-viral vector in an amount of about 1.76×10⁹ copies of the vector/μl to about 1.76×10¹¹ copies of the vector/W, about 1.76×10⁹ copies of the vector/μl to about 1.76×10¹⁰ copies of the vector/μl, or about 1.76×10¹⁰ copies of the vector/μl to about 1.76×10¹¹ copies of the vector/μl.

In embodiments, suitable water channel proteins include, but are not limited to, aquaporins. In an embodiment, the water channel is aquaporin-1. In embodiments, suitable water channel proteins include human aquaporins. In an embodiment, the water channel protein is human aquaporin-1.

In embodiments, the ultrasound may be applied at a frequency of about 0.5 MHz to about 2.5 MHz, or about 1 MHz to about 2 MHz.

In certain aspects, this disclosure describes methods of gene therapy using non-viral vectors. Such non-viral vectors may be employed effectively at relatively low concentrations of vector. Each of these aspects—the non-viral nature of the vector and the low dosage that may be used effectively—may contribute to the increased safety and effectiveness of the treatment. The use of non-viral vectors as described herein may further enable the use of methods that include repeated treatments provided to a given patient, which may increase efficacy while maintaining the safety of the treatment.

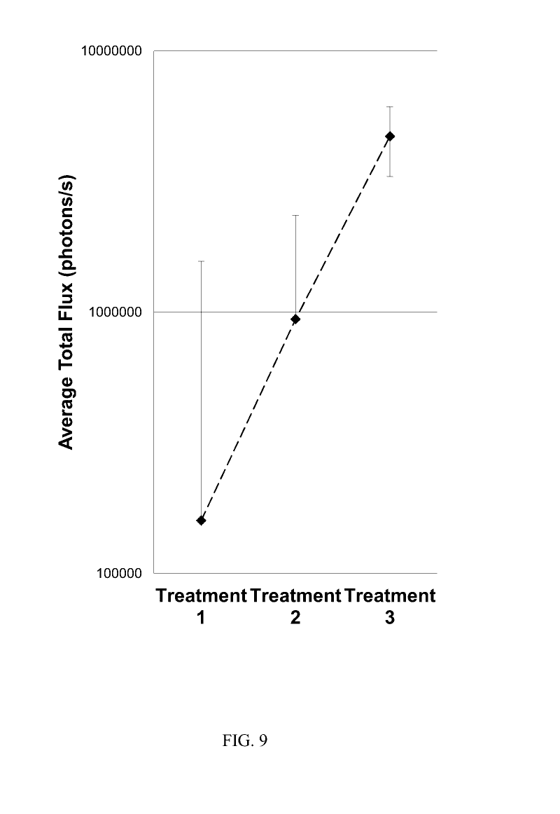

The results shown for Example (FIG. 9) demonstrate the synergistic effects of serial dosing using UAGT for transfer of a non-viral vector. UAGT may be used to titrate gene therapy for human treatment, enabling the development and use of dosing regimens tailored to the needs of a specific patient.

In certain embodiments, the methods described herein may be used to develop a personalized dosing regimen that tailors treatment to a particular patient. In embodiments, such a dosing regimen may include repeated treatments using UAGT. Such a patient-specific dosing regimen may maximize the effectiveness of the treatment while reducing unwanted side effects. As used in this document, with respect to human patients, “xerostomia” relates to the subjective feeling of oral dryness. The subjective nature of xerostomia may enable the therapy to be titrated to the patient's condition. Additionally, the synergistic nature of multiple UAGT treatments may increase the effectiveness of serial dosing while maintaining the safety of the treatment.

In embodiments, there is provided a method of treatment for xerostomia comprising:

A) providing to a salivary gland of a subject in need thereof a UAGT treatment comprising administering a composition comprising microbubbles and a non-viral expression vector comprising a nucleic acid sequence encoding a water channel, and applying ultrasound to the salivary gland;

B) assessing the level of xerostomia in the subject using a visual analog scale (VAS);

C) if the assessment indicates that xerostomia remains present in the subject, providing further treatment to the salivary gland, wherein the treatment comprises administering a composition comprising microbubbles and a non-viral expression vector comprising a nucleic acid sequence encoding a water channel, and applying ultrasound to the salivary gland; and

D) optionally, repeating the above steps A-C until assessment by visual analog scale indicates that xerostomia has been relieved in the subject.

In embodiments, suitable water channel proteins include, but are not limited to, aquaporins. In an embodiment, the water channel is aquaporin-1. In embodiments, suitable water channel proteins include human aquaporins. In an embodiment, the water channel protein is human aquaporin-1.

In embodiments, the non-viral vector is present in the composition at a concentration of about 0.01 μg/μl to about 1 μg/μl, 0.01 μg/μl to about 0.2 μg/μl, about 0.01 μg/μl to about 0.1 μg/μl, or about 0.1 μg/μl to about 1 μg/μl. In embodiments, the composition comprises the non-viral vector in an amount of about 1.76×10⁹ copies of the vector/μl to about 1.76×10¹¹ copies of the vector/W, about 1.76×10⁹ copies of the vector/μl to about 1.76×10¹⁰ copies of the vector/μl, or about 1.76×10¹⁰ copies of the vector/μl to about 1.76×10¹¹ copies of the vector/μl.

In embodiments, the level of xerostomia is assessed at about 1 week after a treatment with UAGT.

In embodiments, the level of xerostomia is assessed at about 1 week to about 2 weeks after a treatment with UAGT.

In embodiments, the above steps may be repeated until xerostomia has been assessed to have been relieved in the subject.

In embodiments, one, two, three, four, five, six, seven, eight, nine, or ten treatments may be provided.

The therapeutic efficacy of UAGT has been demonstrated at a dose of 1 μg/μl for a single treatment. In embodiments, at a lower dose, multiple combined treatments may be used to achieve the effect of the higher dose. Additionally, in view of the synergistic effects observed for UAGT treatments, fewer lower doses may achieve an effect as great as or greater than a single high dose.

The following examples serve to further illustrate various embodiments of the present invention.

### EXAMPLE 1

### Materials and methods

### Animals and Husbandry

The Institutional Animal Care and Use Committee of Allegheny Singer Research Institute approved all animal experimentation described herein. Yucatan miniature swine aged approximately 10 weeks were certified free of common swine diseases and vaccinated for Haemophilus, erysipelas, PCV and mycoplasma.

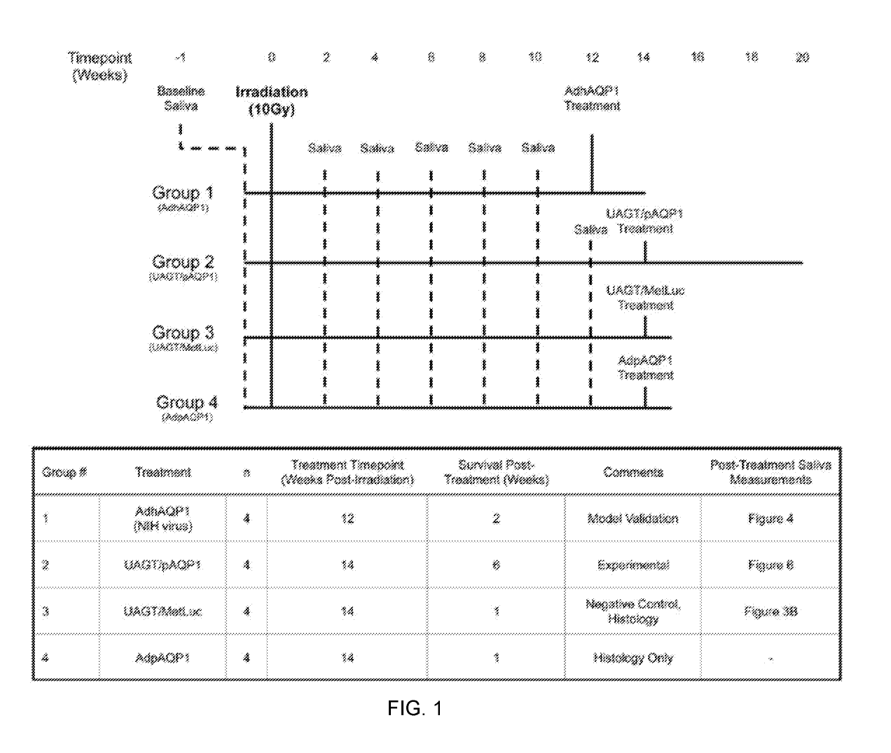

Animals were group housed in large pens, with temperature maintained at 21-26° C. and 30-70% humidity under positive pressure. Enrichment with toys and human contact was provided. Subjects were fed twice daily, according to the breeder's recommendations and allowed free access to water via an automatic system. Subjects acclimated to the colony for one week before being entered into the study. Experimental group assignments are outlined in FIG. 1. Group sizes were based upon pilot studies indicating that n=4 gave a significant effect in gene therapy-treated animals. Animals received a name designation and were randomly assigned to their experimental group upon receipt in the facility. Electron beam treatment planning and irradiation

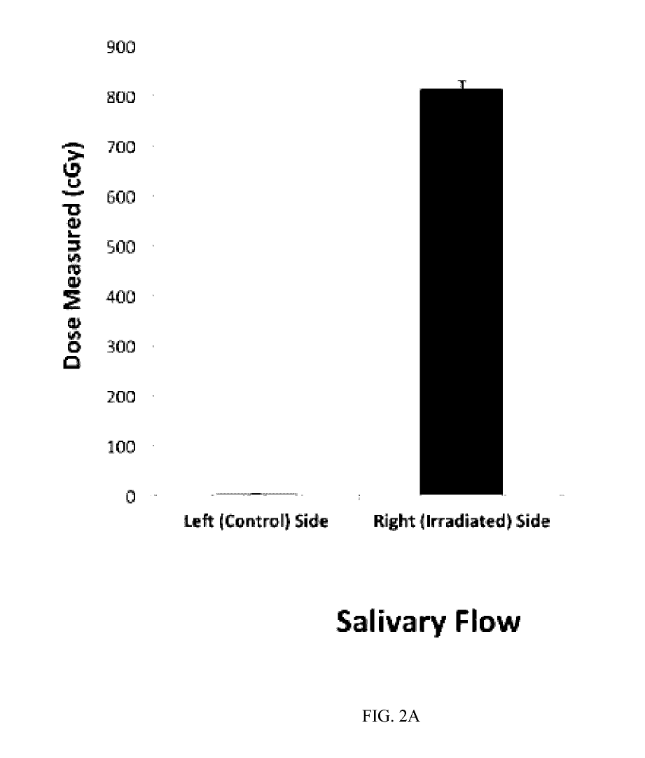

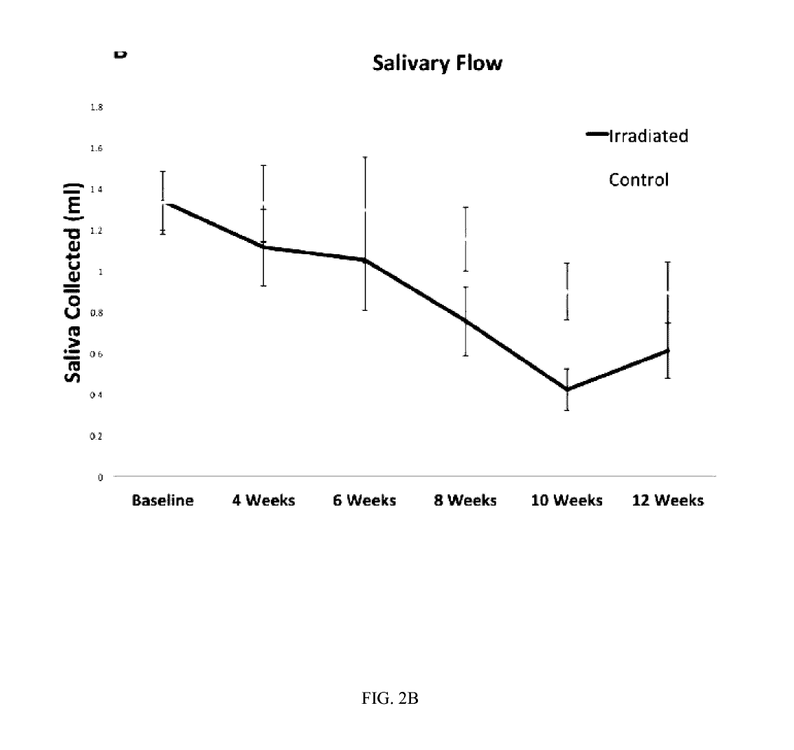

Earlier studies (data not shown) utilizing a 20 Gy electron beam revealed extremely severe damage to the parotid gland, well in excess of that experienced by most patients suffering from radiation-induced xerostomia. A single 10 Gy dose of electrons was calculated to be approximately bioequivalent to the threshold dose for parotid gland dysfunction. The functional deficits reported in FIG. 2B validate this mild dose of radiation as being sufficient to elicit profound functional deficits in the parotid glands of swine subjects.

A treatment plan was devised by computerized tomographic scanning of a single minipig of similar size and weight to all others in the study. This animal subject was used as a model for a vacuum formed bag to ensure targeting accuracy and immobilization of all subjects. This vacuum formed bag was subsequently used for immobilization of all animals participating in the study. CT scanning of this animal was performed using a Siemens SOMATOM SENSATION scanner (Concord, Calif., USA), and radiation treatment planning was performed on a Computerized Medical Systems XIO (Maryland Heights, Mo., USA) planning system.

Irradiation of the right parotid gland was performed in all animal subjects using a Siemens PRIMUS (Concord, Calif.) linear accelerator. Animals were sedated and placed in the vacuum form bag in the linear accelerator. A 12 MeV electron beam calculated at the 90% isodose line was used to treat the gland, exempting a small portion of the anteromedial region to avoid unintended irradiation of the contralateral gland due to the anatomic proximity of the two structures in this area. CERROBEND (Bolton Metal Products, Bellefonte, Pa.), an eutectic alloy comprised of 50% bismuth, 26.7% lead, 13.3% tin, and 10% cadmium by weight was used to block the electron beam from surrounding structures following custom anatomic fitting to match the right swine parotid gland. A 1.0 cm margin around the target gland was added to the aperture of the block to allow for respiratory motion and to allow build-up of electron dose. Following irradiation and recovery from sedation, all animals were returned to the husbandry unit without event.

### Saliva Collection

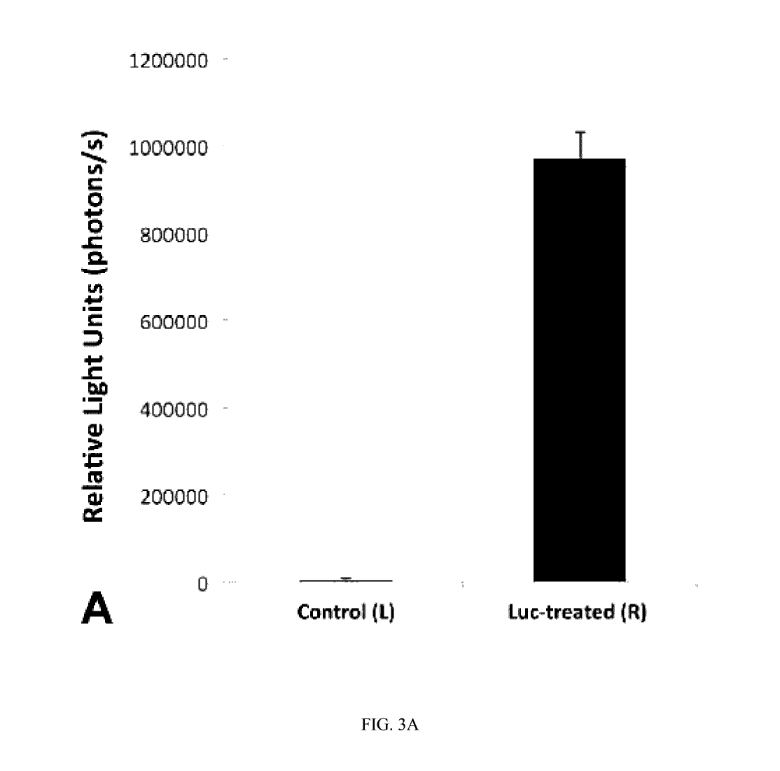

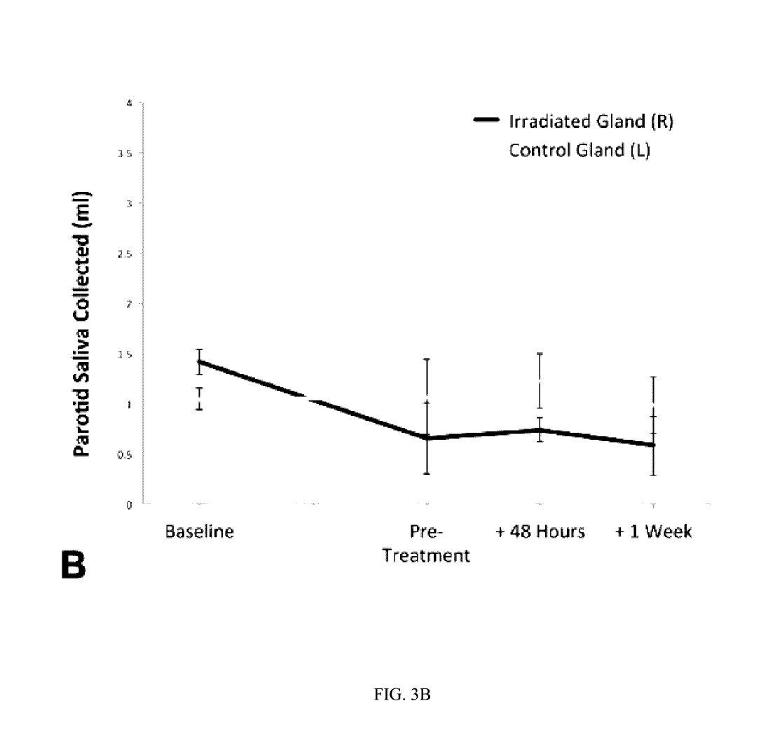

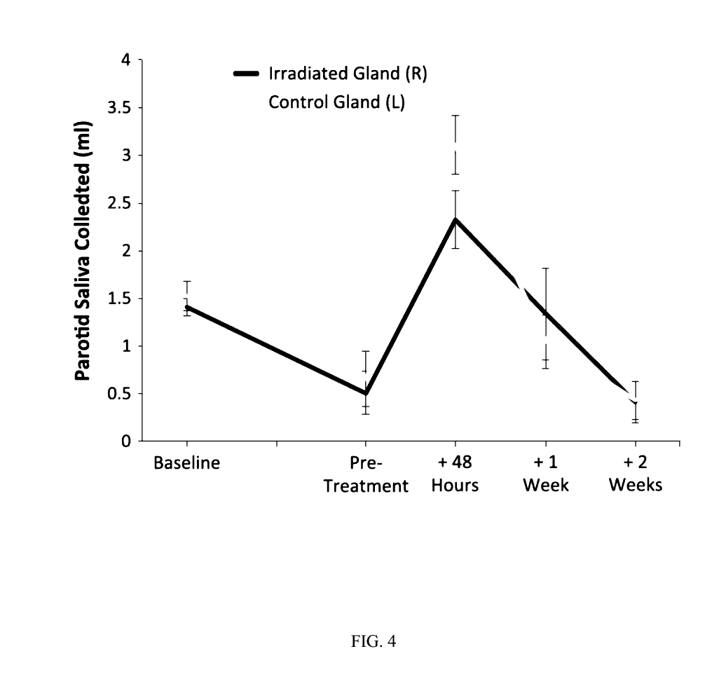

All saliva collections were performed between 6:30 am and 10:00 am, and the time points of saliva collection are shown in the respective figures, including FIG. 2B, FIG. 3B, FIG. 4, and FIG. 6. No blinding was utilized in this study. Animals were initially sedated by intramuscular injection of a mixture of ketamine (20 mg/kg) and xylazine (2 mg/kg), then placed on an operating table, intubated, and place in the prone position, maintained with isoflurane anesthesia (1-1.5%) via artificial ventilation. An intramuscular injection of 1 mg/kg of Pilocarpine was given and 10-15 minutes were allowed to elapse for the sialogogue to take effect (determined by pooling of saliva in the anterior floor of the mouth). An oral swab with a 2 mL capacity (Salimetrics, Carlsbad, Calif., USA) was weighed and then placed over the opening of Stensen's duct, in the buccal corridor and mechanically secured. Saliva was collected by capillary action for 10 minutes, and the oral swab was replaced if it appeared to be approaching capacity. After 10 minutes, the oral swab(s) were weighed and saliva volume calculated by subtracting initial weight from final weight, assuming a specific gravity of 1 for the saliva. Saliva was removed from the oral swab by centrifugation at 3000 rpm for 15 minutes at 4° C. and analyzed as described.

pAQP1 Construction and Testing

Porcine (Sus scrofa) AQP1 (NCBI, NM 414454) was synthesized by Integrated DNA Technologies (Coralville, Iowa, USA). The cDNA was amplified with the following primers: Forward: 5′-ATAGGATCCACCTGGCCAGCGAGTTCAAGAAGAAG (SEQ ID NO:1), Reverse 5′-TATCTCGAG TTATTTGGGCTTCATCTCCAC (SEQ ID NO:2) and cloned into pCMV-MCS (Agilent Technologies, Santa Clara, Calif., USA) using BamHI and XhoI restriction sites, resulting in pCVM-pAQP1.

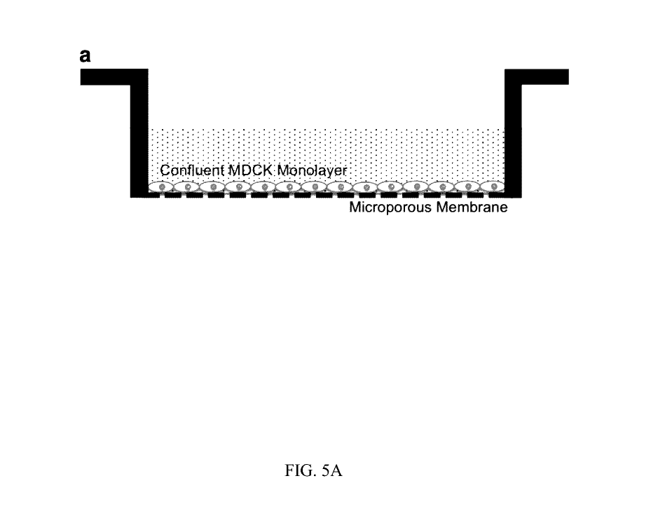

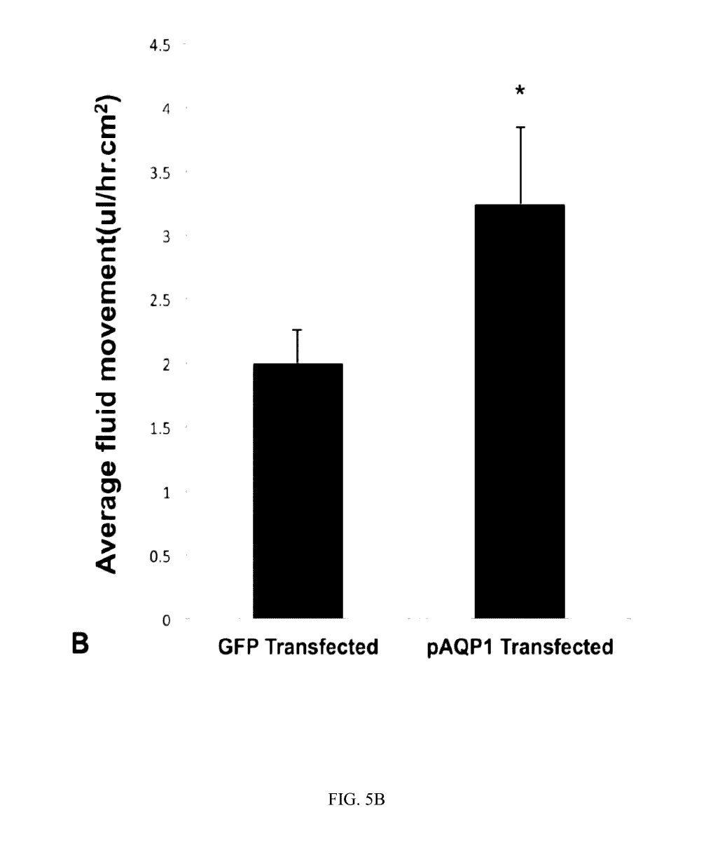

pAQP1 channel function was tested by expressing the pCMV-pAQP1 plasmid in Madin-Darby Canine Kidney epithelial (MDCK) cells (Source: ATCC). Transfection was performed using Lipofectamine 2000 (Life Technologies, Carlsbad, Calif., USA), using a (Green Fluorescent Protein (GFP)-expressing plasmid in parallel cultures as a control for transfection efficiency. 48 hours after transfection, cells were seeded on collagen-coated polycarbonate filter inserts in six-well plates (Corning, Corning, N.Y., USA) and placed in a transwell system as shown in FIG. 5A. After cells formed a confluent monolayer, the apical chamber was replaced with 1.5 ml hyperosmotic sucrose/(Dulbecco's Modified Eagle Medium) DMEM medium (440 mOsmol/L) and the medium in the basal chamber was replaced with fresh 2.6 ml DMEM medium. After 48 hr, the fluid volume in the individual apical chambers was measured by pipette and the transepithelial net fluid movement was calculated relative to GFP controls.

### Adenoviral Vector Construction, Purification, and Testing

The AdhAQP1 was obtained as an aliquot of the clinically-validated vector. This vector was not further tested prior to upscaling. The vector was upscaled in HEK293 cell cultures, and purified using 2× CsCl gradient centrifugation. Viral titer (vp) was determined using optical absorbance.

To construct a recombinant adenovirus encoding porcine AQP1, the pAQP1 cDNA was amplified from pCMV-pAQP1, described above, using the following primers: Forward: 5′-GCTCGAGCCTAAG TTCCACCATGGCCA CGAGTCAGGAAG (SEQ ID NO:3), Reverse 5′-TCTTATCTAGAAGCTTTTATTTGGGCTTCATCTCCAC (SEQ ID NO:4). The cDNA was cloned into the HindIII restriction site of the pShuttle-CMV vector (Agilent Technologies, Carlsbad, Calif., USA) using the In-Fusion cloning system. (Clontech, Mountain View, Calif., USA). The resulting pShuttle-pAQP1 was linearized by digestion with PmeI and transformed into E. coli; BJ 5183-AD-1 strain cells. (Agilent Technologies). Recombinant clones was selected by kanamycin resistance and confirmed by PacI endonuclease restriction analysis. Finally the linearized recombinant plasmid was transfected into HEK293 cells by polyethylenimine transfection. The primary viral stock was prepared by freeze/thaw cycles of cells 15 days post-transfection. The AdpAQP1 was upscaled and purified as described above and functionally tested using the transwell system described for the pCMV-pAQP1 plasmid.

### Gene Transfer to the Salivary Gland

All gene transfer procedures were performed between 6:30 am and 10:00 am. The animals were sedated, intubated, and positioned as described under Saliva Collection above, with the exception that animals are placed supine for cannulation of Stensen's duct. An intramuscular injection of 0.54 mg atropine was given and 10 minutes were allowed to elapse for the drug to take effect. A P50 catheter fused to a P10 catheter was inserted into the opening of Stensen's duct on the right side and secured with Vetbond (3M Products, St. Paul, Minn., USA). All infusions were made using 3 ml of sterile phosphate-buffered saline and the tubing left in place for 10 minutes following infusion. For Adenoviral gene delivery, 1×10¹⁰ viral particles of AdhAQP1 or AdpAQP1 were diluted in the infusate. All gene transfer studies were carried out on the right parotid gland only, and the left (control) gland was never cannulated.

For UAGT, DEFINITY microbubbles (Lantheus Medical Imaging, North Billerica, Mass.) were activated per manufacturer's instructions immediately prior to the procedure and mixed with 1 mg/ml of the plasmid vector (either pCMV-pAQP1 or pCMV-MetLuc) before being added to the infusate (total dose 3 mg).

Immediately after infusion, a layer of clinical ultrasound gel was applied to the skin overlying the parotid gland, and SoniGene ultrasound beam emitter (VisualSonics, Toronto, Canada) was applied to the skin and moved over the outline of the parotid gland using a gliding motion. A total ultrasound exposure of 4 treatments comprising 30 seconds each, 2 W/cm² at a 50% duty cycle with 10 seconds between treatments was delivered. Following gene transfer, the animal was allowed to awaken and returned to its housing.

### Tissue Processing and Histological Analysis

Parotid glands were dissected from the specimen, partially section across the transverse plane (i.e. “bread loafed”) to facilitate fixation, and immediately fixed in 20 volumes of formalin per weight. The glands were examined and at least 5 representative sections were harvested per each gland. Tissue sections were processed for conventional H&E staining.

### Statistical Analysis

For statistical comparisons of measurements at a single time point, including FIGS. 2A, 3A, and 5B, an unpaired, single-tailed t-test was utilized. For statistical comparisons of multiple saliva measurements made over time, including FIGS. 2B, 3B, 4, and 6, a repeated Measures ANOVA with mixed effects using a compound symmetry variance and post-hoc Tukey adjusted differences of least squares mean was utilized. In all analyses, p values of <0.05 were considered significant.

### Proteomic Profiling of Swine Saliva

Proteomic profiling was performed on pig saliva using methods similar to those described by Geguchadze R, et al. (Molecular Therapy—Methods and Clinical Development. 2 Apr. 2014). Briefly, a clean-up step was performed using a 2-D Quant kit (GE Healthcare, Little Chalfont, Buckinghamshire, UK) and dissolved using 2D gel rehydration buffer with 50 mM Tris pH 8.5. Protein concentrations were evaluated using Bradford method and samples were appropriate diluted to a final protein concentration of 1μg/μl. 1 μl of Cy3 or Cy5 NETS-conjugated dyes (Lumiprobe, Inc., Hallandale Beach, Fla., USA) were added to 50 μg of protein sample and incubated on ice for 30 min in the dark. 1 μl of 10 mM lysine was used to quench the reaction by incubating on ice for 10 min in the dark.

2-D SDS gels were run mixing 10 μg of each paired sample and diluting further in rehydration buffer to 450 μl and placing on a 24 cm isoelectric focusing (IEF) strip (pH3-10NL). These strips were previously rehydrated using 2% DTT (dithiothreitol), 0.5% IPG (GE Healthcare) buffer and 0.002% bromophenol blue at RT for 8 hours. Rehydrated strips were loaded on Ettan IPGphor 3 IEF system, samples were placed, and run overnight for a total of 60,000 volt-hours (vhr) and equilibrated with 1% DTT followed by 2.5% iodoacetamide for 15 min each. The second dimension was carried out in a homogeneous 13.5% SDS gel.

Completed gels were scanned using a Typhoon 9400 scanner. The pictures were edited using ImageQuant TL 7.0 software and Differential In-gel Analysis (DIA) and Biological Variation Analysis (BVA) of the 2D DiGE results were performed using DeCyder 2D 7.0 Software (GE Healthcare). All protein spots-of-interest, identified by BVA and DIA analyses, were manually checked to insure that they were not changed in the Baseline versus Control gels, and thus the decrease or loss of these proteins can be definitively attributed to the effects of irradiation and/or gene therapy. Spots identified as being significantly decreased in the Baseline versus UAGT/pAQP1 gels but unchanged in the Baseline versus Control gels were extracted using an Ettan Spot Picker (GE Healthcare).

Protein identification was performed as described by Wu, et al. (Wu C, et al. Molecular & Cellular Proteomics: MCP. 2010 October; 9(10):2262-75). Briefly, protein spots of interest from 2D gels were excised, reduced with DTT and alkylated with iodoacetamide, digested with trypsin and desalted with C18 ZipTips (Millipore, Billerica, Mass., USA). Both MS and MS/MS analyses of the digested peptides were performed on a MALDI-TOF/TOF tandem MS (Bruker UltrafleXtreme, Bruker Daltonics Inc. Billerica, Mass., USA). The database search and analysis were performed using FlexAnalysis and BioTools software (Bruker Daltonics Inc.) against “Other Mammalia (excluding primate and rodents)” Swiss-Prot protein database using a local Mascot server.

### EXAMPLE 2

A single 10 Gy dose of radiation delivered by an electron beam to the right parotid gland results in significant, sustained, and bilateral hyposalivation in the miniature swine.

To confirm that the control gland received no radiation, thermo-luminescent detectors were placed on the skin overlying the right (treated) and left (control) parotid gland during the application of the electron beam. FIG. 2A illustrates the radiation dose measured on the skin overlying the left (control) and right (irradiated) parotid glands and shows that the control gland received no detectable radiation. Error bars are +/−SEM.

A baseline stimulated, isolated parotid saliva collection was performed on all animals (n=16), bilaterally and immediately prior to irradiation (approximately 10 weeks of age) and at 4, 6, 8, 10, and 12 weeks post-irradiation. Saliva volume was determined by weight, assuming a specific gravity of 1.0. FIG. 2B illustrates stimulated isolated parotid saliva volumes at pre-irradiation baseline and for the irradiated (right) and control (left) sides at various time points post-irradiation in the 16 subjects participating in this study. Error bars are +/−SEM. Notably, despite the demonstrated lack of radiation damage to the control gland, saliva output of the control side began to decline from baseline by Week 8, decreasing further at Week 10 and Week 12.

Statistical analysis determined that significant differences existed between irradiated and control volumes (p=0.0003) but no interaction between the covariates suggesting both groups changing over time at similar rates. Evaluating the treatment status versus baseline across all weeks indicated that the irradiated side was significantly different than baseline (p=0.0002) but the control side was not (p=0.5).

The conservative statistical strategy did not find significant differences between Baseline and Control to support the visually obvious trend, but suggested Control and Irradiated were changing over time at similar rates. These findings indicate a functional coupling of the right and left parotid glands. While not intending to be bound by any theory of operation, it is believed that this may have a neurological basis.

### EXAMPLE 3

Ultrasound-assisted gene transfer of met-Luc to the irradiated salivary gland produces bioluminescent saliva but no increase in stimulated saliva volume.

Ultrasound-assisted gene transfer via “sonoporation” has been reported in the mouse salivary gland, but it is believed that the application of this technology to the salivary gland of the swine has never been reported previously. Demonstrating this phenomenon in the pig proved challenging since the size of the animal precludes the use of the conventional IVIS imaging technique previously relied upon. The present inventor therefore turned to the secreted Metridia longa luciferase (MetLuc) reporter gene, reasoning that if this reporter gene was expressed in the pig's salivary gland, luminescence would be detected in the saliva secreted from a salivary gland thus treated.

FIG. 3A shows the results of a MetLuc assay performed on stimulated saliva collected from the Irradiated/MetLuc-treated (right) side 48 hours after UAGT, compared with the control (left) side. Relative light units (photons/s) measured from stimulated saliva samples taken 48 hours following UAGT of a MetLuc-expressing plasmid to the right (R) parotid gland (n=4). The difference between luminescence of saliva from right and left (L) was highly significant (p<0.01). Error bars are +/−SEM. These results demonstrate, for the first time, ultrasound-assisted non-viral gene transfer to the salivary gland of adult swine and also illustrate the precise isolation of saliva collected from individual parotid glands, with no detectable mixing of samples from the treated (right) and control (left) glands.

This experiment served a second purpose; that of a negative control for ultrasound-assisted AQP1 gene therapy, described below. Pigs receiving MetLuc via UAGT underwent exactly the same manipulation as animals receiving either Adenoviral or ultrasound-assisted AQP1 gene therapy as described below. FIG. 3B shows stimulated parotid saliva volumes at Baseline, Pre-Treatment, and 48 hours and 1 week following UAGT of MetLuc. Error bars are +/−SEM. Stimulated saliva volumes collected immediately prior to, and 48 hours after, UAGT with MetLuc, demonstrate that there is no effect of the UAGT methodology per se, on stimulated parotid saliva volume.

### EXAMPLE 4

Xerostomia in a swine model is reversed by the clinically validated Adenovirus expressing human AQP1 (hAQP1).

The intent of this study was to measure AQP1 gene therapy delivered by UAGT against the current clinical standard, AQP1 delivered by Adenovirus. Human AQP1, delivered by a viral vector, has previously been utilized as a gene therapy for xerostomia in the rat, the swine, and ultimately humans. Thus, it was investigated whether a swine model of radiation-induced xerostomia responds to Adenoviral gene therapy in a manner consistent with what was previously reported by Shan et al. (Shan Z, et al. Mol Ther. 2005 March; 11(3):444-51. Epub 2005/02/25. eng.). A non-clinical grade prep of the AdhAQP1 virus was used to ensure that the same vector was used that has shown therapeutic efficacy in the human clinical trial.

FIG. 4 shows stimulated parotid saliva volumes at Baseline, Pre-Treatment (12 weeks post-irradiation), and 48 hours and 1 week and 2 weeks following gene therapy with 1×10¹⁰ vp of AdhAQP1 (n=4). Error bars are +/−SEM. Consistent with earlier preclinical work utilizing AdhAQP1 in the swine model the animals received 1×10¹⁰ viral particles (vp) of the vector to the irradiated gland, 12 weeks following irradiation (n=4). The results demonstrate that AdhAQP1 reverses hyposalivation in the swine model in a manner similar to that reported by Shan et al.

Statistical analysis determined that no significant differences existed between irradiated (R) and control (L) gland saliva volumes (p=0.4) suggesting both groups change together. Comparisons between weeks noted the following significant differences: Baseline versus Pretreatment (p=0.01), Pretreatment versus +48 Hours (0.0001), +48 Hours versus +1 Week (p=0.0001).

Gene therapy delivered to the irradiated gland was observed to substantially enhance stimulated fluid secretion from the contralateral gland. The extinction of the therapeutic effect within two weeks is consistent with the known gene expression dynamics of the Adenoviral vector in mammals.

### EXAMPLE 5

Porcine AQP1(pAQP1) expressed in MDCK cells results in transcellular water permeability.

The above experiment in Example 4 demonstrates that the swine model responds to AdhAQP1 gene therapy in a manner similar to the earlier swine model that utilized unilateral irradiation with photons. However, since UAGT or “sonoporation” gene transfer technology obviates the need for a viral vector, and thus avoids the introduction of foreign antigens into the damaged salivary gland, it was decided to carry experiments forward with porcine AQP1 (pAQP1).

pAQP1 was cloned as described in Material and Methods and expressed by plasmid transfection into MDCK canine kidney epithelial cells plated as a monolayer in transwell chambers. Compared to GFP-transfected cells, cells transfected with pAQP1 showed enhanced water flux across the monolayer. These experiments demonstrate the physiological functionality of the pAQP1 transgene product.

FIGS. 5A-B illustrate the testing of pAQP1 channel function. FIG. 5A shows a schematic of the transwell culture system utilized to assess transcellular fluid flux across a confluent MDCK monolayer. The upper layer of the well is 440 mOsmol/L hyperosmotic sucrose/DMEM media and the lower layer is standard DMEM media. FIG. 5B shows quantification of transcellular flux in GFP-transfected and pAQP1-transfected MDCK cells over a 48 hour period. * indicates statistically significant (p<0.05) difference between GFP- and pAQP1-transfected cells. Error bars are +/−SEM.

### EXAMPLE 6

Porcine AQP1 gene therapy delivered to the irradiated salivary gland with UAGT increases stimulated salivary flow to levels comparable with Adenoviral gene therapy.

It was investigated whether UAGT of pAQP1 to the irradiated gland can increase stimulated parotid saliva volume in a fashion similar to that previously demonstrated with hAQP1 delivered by an Adenovirus. FIG. 6 illustrates pAQP1 gene therapy using UAGT in an irradiated pig model. Stimulated parotid saliva volumes are shown at Baseline, Pre-Treatment (14 weeks post-irradiation), and 48 hours, 1 week, 2 weeks, 3 weeks, 4 weeks, 5 weeks and 6 weeks following gene therapy with UAGT/pCMV-AQP1 (n=4). Error bars are +/−SEM. Increases in stimulated parotid saliva volume were observed in both treated and control sides in UAGT/pAQP1 treated-animals (n=4, FIG. 6), with the magnitude of the increase being roughly comparable to that seen with the AdhAQP1 virus in this model. A bilateral effect was also observed, despite the fact that gene therapy was delivered only to the irradiated side.

Statistical analysis determined that no significant differences existed between irradiated and control volumes (p=0.0009) but no interaction between the covariates suggesting both groups changing over time at similar rates. Comparisons between weeks noted the following significant differences: Baseline versus Pretreatment (p=0.06), Pretreatment versus +48 Hours (0.06), +48 Hours versus +1 Week (p=0.004).

The increases in saliva volume declined substantially by 2 weeks post-treatment, showing a trend toward persistent therapeutic effect on the control side but no longer statically different from pre-treatment levels. Notably, function improved markedly in the control gland despite the fact that gene therapy was only delivered to the irradiated gland.

### EXAMPLE 7

UAGT does not promote the local inflammation observed with Adenoviral gene transfer to the salivary gland.

A major factor limiting the clinical utility of Adenoviral gene therapy in chronic conditions like xerostomia is the vector's robust immunogenicity; a phenomenon that has previously been described histologically in the salivary gland, even with UV-inactivated Adenoviral particles, suggesting it is mediated entirely by the viral capsid. Further, systemic inflammation resulting from Adenoviral gene transfer to the salivary gland, as indicated by chronic inflammatory focal lesions and induction of anti-Adenoviral antibodies, has also been reported. As the present UAGT technique accomplishes gene therapy that is comparable to Adenovirus-mediated gene transfer, but obviates the introduction of viral antigens into the subject's body, it was believed that UAGT would also minimize local and systemic inflammatory response.

To test this hypothesis, an Adenoviral vector was constructed expressing the pAQP1 cDNA described above. A group of swine (n=4, Group 4), irradiated on the right side as described above, were treated with this AdpAQP1 and this group was sacrificed 1 week after gene transfer. The UAGT/MetLuc negative control animals (n=4, Group 3), described previously, were sacrificed 1 week after gene transfer and served as the UAGT control. In all experiments, the unirradiated (left) parotid gland was left untouched and served as an internal control for each animal. The design of this experiment was such that the effects of Adenoviral gene delivery relative to UAGT could be determined upon the histology of the parotid gland.

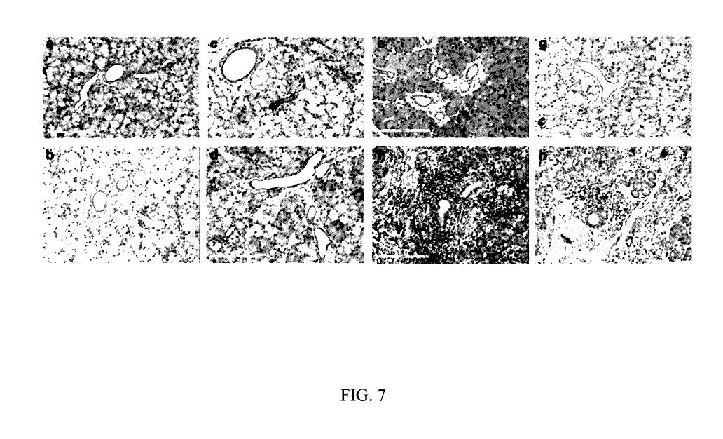

Slides were reviewed by an oral pathologist blinded as to experimental design and animal identities. Results of this experiment are summarized in Table 1 and representative sections are shown in FIG. 7. FIGS. 7A-H show representative histology of parotid glands 1 week following Adenoviral gene transfer and UAGT. The top row (A, C, E, G) are contralateral, non-irradiated control glands from the corresponding treated animals in the bottom row (B, D, F, H). B and D are UAGT/pAQP1-treated and F and H are AdpAQP1 treated. Scale bars are 200 μm.

Relative to control glands, two primary histological findings were noted. First, mild acinar cell pleomorphism and changes in zymogen granules were observed in all irradiated glands (FIG. 7 panels 7B, 7D, 7F, 7H). The second finding was interstitial and periductal inflammation, which was restricted to glands treated with the Adenoviral vector (FIG. 7 panels 7F, 7H). Importantly, UAGT had no detectable effect upon the structure or morphology of the salivary gland, reinforcing the safety of this technique relative to the Adenoviral vector.

TABLE 1
Blinded expert histological analysis of parotid glands
1 week following Adenoviral gene transfer and UAGT

| | | | | | |
|---|---|---|---|---|---|
| 9 | Group 3 | R | Irradiation, | Swelling, vacuolization, zymogen | Minimal periductal |
| | | | UAGT/MetLuc | granule pleomorphism | lymphocytic |
| | | | | | infiltration |
| | | L | None | Normal | [symbol] |
| 10 | | R | Irradiation, | Swelling, vacuolization, zymogen | Normal |
| | | | UAGT/MetLuc | granule pleomorphism | |
| | | L | None | Normal | Normal |
| 11 | | R | Irradiation, | Swelling, vacuolization, zymogen | Normal |
| | | | UAGT/MetLuc | granule pleomorphism | |
| | | L | None | Normal | Normal |
| 12 | | R | Irradiation, | Swelling, vacuolization, zymogen | Normal |
| | | | UAGT/MelLuc | granule pleomorphism | |
| | | L | None | Normal | Normal |
| 13 | Group 4 | R | Irradiation, | Swelling, vacuolization, zymogen | Moderate periductal |
| | | | AdpAQP1 | granule pleomorphism | fibrosis, moderate |
| | | | | | periductal lymphocytic |
| | | | | | infiltration, |
| | | | | | salivary ductal ectasia |
| | | L | None | Normal | |
| 14 | | R | Irradiation, | Swelling, vacuolization, zymogen | Moderate periductal |
| | | | AdpAQP1 | granule pleomorphism | fibrosis, moderate |
| | | | | | periductal lymphocytic |
| | | | | | infiltration, perivascular |
| | | | | | sclerosis |
| | | L | None | Normal | |
| 15 | | R | Irradiation, | Swelling, vacuolization, zymogen | Mild periductal |
| | | | AdpAQP1 | granule pleomorphism, focal | fibrosis, moderate |
| | | | | atrophy | periductal lymphocytic |
| | | | | | infiltration |
| | | L | None | Normal | |
| 16 | | R | Irradiation, | Swelling, vacuolization, zymogen | Normal |
| | | | AdpAQP1 | granule pleomorphism | |
| | | L | None | Normal | Normal |

[symbol]

indicates data missing or illegible when filed

### EXAMPLE 8

Saliva produced by the damaged parotid gland after gene therapy shows a profile very similar to normal saliva, but with the loss of select proteins of putative acinar origin.

Aquaporin-1 gene therapy as a treatment for radiation-induced xerostomia uses the mechanism of transcellular movement of fluid via AQP1 across surviving ductal cells. This is not fundamentally different from normal physiological salivation, which utilizes primarily Aquaporin-5 to drive transcellular fluid movement across acinar cells. In both instances, the source of the bulk of this fluid is ultimately the interstitium, which in turn is mainly dependent upon the serum for its protein composition. Accordingly, the degree to which the proteome of “saliva” produced as a result of gene therapy would differ from natural saliva was explored.

To investigate this issue, proteomic profiling was performed using difference gel electrophoresis (DiGE) on matched samples of saliva obtained 48 hours after gene therapy from the right (irradiated, treated) and left (control, untreated) parotid glands within the same animal relative to baseline saliva collected and banked from the same animal prior to irradiation. In order to evaluate the quality of this saliva in the most clinically meaningful way, saliva obtained from the treated and control parotid glands were indexed to baseline saliva.

FIGS. 8A-8C show proteomic profiling of pooled saliva obtained from and pre-injury baseline (A), contralateral control glands (B) and irradiated, UAGT/pAQP1 gene-therapy-treated parotid glands (C) (n=4/group) 48 hours after UAGT treatment. Each image is a grayscale representation of fluorescent intensity of a pooled sample of each group, each labeled with a different Cy dye (Cy2, Cy3, Cy5). Pooled samples were run simultaneously on the same gel, and groups were differentiated by their Cy dye and images corrected to compensate for differences in dye fluorescence intensity. These profiles were generated for visual purposes and were not utilized for statistical analysis. The global similarity of Baseline and Control saliva relative to UAGT/pAQP1 saliva is visually evident (FIGS. 8A and 8B versus 8C). This indicates that the saliva produced by the contralateral gland in response to UAGT treatment is likely to have a normal complement of salivary proteins.

Biological Variation Analysis (BVA) was performed using the DeCyder platform, and found the following differences: Baseline versus Control, 152 of 2253 matched spots significantly changed (p<0.05), and Baseline versus Treated, 186 of 2362 matched proteins significantly changed (p<0.05). In order to evaluate the potential clinical significance of salivary proteins lost as a result of radiation injury to the parotid gland, spots were selected and extracted that were determined to be significantly altered in UAGT/pAQP1 saliva versus Baseline saliva, but not significantly altered in Control versus Baseline saliva (a manual step confirmed this as a check on the software). Of the 108 spots extracted, 71 were positively identified using mass spectrometry, and results are shown in Table 2.

Table 2 shows the identities of proteins determined to be significantly reduced in saliva obtained from irradiated, UAGT/pAQP1-treated parotid glands relative to saliva obtained at the same time from contralateral, control glands. Data are separated according to their database identification, either from the swine database, or the other mammals database.

TABLE 2

| | | |
|---|---|---|
| | | Fold- |
| | | Change |
| | | (AQP1- |
| | | treated/ |
| Accession # | Description | Baseline) |

Pig Database

| | | |
|---|---|---|
| CP8B1_PIG | 5-beta-cholestane-3-alpha,7-alpha-diol 12-alpha-hydroxylase OS = Sus | |
| | scrofa GN = CYP8B1 PE = 2 SV = 1 | −2.2 |
| 5HT1D_PIG | 5-hydroxytryptamine receptor 1D (Fragment) OS = Sus scrofa | |
| | GN = HTR1D PE = 2 SV = 1 | −2.5 |
| ACTS_PIG | Actin, alpha skeletal muscle OS = Sus scrofa GN = ACTA1 PE = 3 SV = 1 | 2.2 |
| ACTB_PIG | Actin, cytoplasmic 1 OS = Sus scrofa GN = ACTB PE = 2 SV = 2 | 2.2 |
| ADML_PIG | ADM OS = Sus scrofa GN = ADM PE = 1 SV = 1 | 2.0 |
| ATPD_PIG | ATP synthase subunit delta, mitochondrial (Fragment) OS = Sus scrofa | |
| | GN = ATP5D PE = 2 | |
| | SV = 1 | 3.4 |
| CAN1_PIG | Calpain-1 catalytic subunit OS = Sus scrofa GN = CAPN1 PE = 2 SV = 3 | 2.2 |
| CHLE_PIG | Cholinesterase (Fragment) OS = Sus scrofa GN = BCHE PE = 2 SV = 1 | −6.1 |
| CO3_PIG | Complement C3 OS = Sus scrofa GN = C3 PE = 1 SV = 2 | 2.9 |
| CP2E1_PIG | Cytochrome P450 2E1 OS = Sus scrofa GN = CYP2E1 PE = 2 SV = 1 | 2.1 |
| HPT_PIG | Haptoglobin OS = Sus scrofa GN = HP PE = 1 SV = 1 | 2.5 |
| LAC_PIG | Ig lambda chain C region OS = Sus scrofa PE = 1 SV = 1 | 3.3 |
| MOES_PIG | Moesin OS = Sus scrofa GN = MSN PE = 2 SV = 3 | 2.7 |
| MYH1_PIG | Myosin-1 OS = Sus scrofa GN = MYH1 PE = 2 SV = 1 | 2.4 |
| OPTN_PIG | Optineurin OS = Sus scrofa GN = OPTN PE = 1 SV = 1 | 2.5 |
| AMYP_PIG | Pancreatic alpha-amylase OS = Sus scrofa GN = AMY2 PE = 1 SV = 3 | 2.9 |
| PECA1_PIG | Platelet endothelial cell adhesion molecule OS = Sus scrofa | |
| | GN = PECAM1 PE = 2 SV = 1 | 2.2 |
| COLI_PIG | Pro-opiomelanocortin OS = Sus scrofa GN = POMC PE = 1 SV = 1 | 2.0 |
| S10AC_PIG | Protein S100-A12 OS = Sus scrofa GN = S100A12 PE = 1 SV = 2 | 3.5 |
| SAL_PIG | Salivary lipocalin OS = Sus scrofa GN = SAL1 PE = 1 SV = 1 | 6.7 |
| TRFE_PIG | Serotransferrin OS = Sus scrofa GN = TF PE = 1 SV = 2 | −3.3 |
| ALBU_PIG | Serum albumin OS = Sus scrofa GN = ALB PE = 1 SV = 2 | 2.1 |
| SDHB_PIG | Succinate dehydrogenase [ubiquinone] iron-sulfur subunit, | |
| | mitochondrial OS = Sus scrofa | |
| | GN = SDHB PE = 1 SV = 1 | 2.0 |
| TPM4_PIG | Tropomyosin alpha-4 chain OS = Sus scrofa GN = TPM4 PE = 2 SV = 3 | 4.5 |
| TRYP_PIG | Trypsin OS = Sus scrofa PE = 1 SV = 1 | 2.3 |
| MYO7A_PIG | Unconventional myosin-VIIa (Fragment) OS = Sus scrofa GN = MYO7A | |
| | PE = 2 SV = 1 | 3.4 |
| UPK2_PIG | Uroplakin-2 OS = Sus scrofa GN = UPK2 PE = 2 SV = 3 | −6.0 |

Other Mammalian Database (excluding Primates and Rodents)

| | | |
|---|---|---|
| 1433B_BOVIN | 14-3-3 protein beta/alpha OS = Bos taurus GN = YWHAB PE = 1 SV = 2 | 5.5 |
| 1433E_BOVIN | 14-3-3 protein epsilon OS = Bos taurus GN = YWHAE PE = 2 SV = 1 | 5.5 |
| 1433F_BOVIN | 14-3-3 protein eta OS = Bos taurus GN = YWHAH PE = 1 SV = 2 | 5.5 |
| 1433G_BOVIN | 14-3-3 protein gamma OS = Bos taurus GN = YWHAG PE = 1 SV = 2 | 5.5 |
| 1433S_BOVIN | 14-3-3 protein sigma OS = Bos taurus GN = SFN PE = 2 SV = 1 | 5.5 |
| 1433T_BOVIN | 14-3-3 protein theta OS = Bos taurus GN = YWHAQ PE = 2 SV = 1 | 5.5 |
| 1433Z_BOVIN | 14-3-3 protein zeta/delta OS = Bos taurus GN = YWHAZ PE = 1 SV = 1 | 5.5 |
| ACTC_BOVIN | Actin, alpha cardiac muscle 1 OS = Bos taurus GN = ACTC1 PE = 2 SV = 1 | 2.2 |
| ACTA_BOVIN | Actin, aortic smooth muscle OS = Bos taurus GN = ACTA2 PE = 1 SV = 1 | 2.2 |
| ACTH_BOVIN | Actin, gamma-enteric smooth muscle OS = Bos taurus GN = ACTG2 | |
| | PE = 2 SV = 1 | 2.2 |
| ADA_BOVIN | Adenosine deaminase OS = Bos taurus GN = ADA PE = 1 SV = 3 | 2.3 |
| SNTA1_BOVIN | Alpha-1-syntrophin OS = Bos taurus GN = SNTA1 PE = 2 SV = 1 | 5.5 |
| AP2M1_BOVIN | AP-2 complex subunit mu OS = Bos taurus GN = AP2M1 PE = 1 SV = 1 | 3.5 |
| CALM_BOVIN | Calmodulin OS = Bos taurus GN = CALM PE = 1 SV = 2 | 2.6 |
| COMT_BOVIN | Catechol O-methyltransferase OS = Bos taurus GN = COMT PE = 2 SV = 1 | 3.5 |
| CHLE_HORSE | Cholinesterase OS = Equus caballus GN = BCHE PE = 1 SV = 1 | −6.1 |
| CFTR_LOXAF | Cystic fibrosis transmembrane conductance regulator OS = Loxodonta | |
| | africana GN = CFTR | |
| | PE = 3 SV = 1 | −2.3 |
| DPP6_BOVIN | Dipeptidyl aminopeptidase-like protein 6 OS = Bos taurus GN = DPP6 | |
| | PE = 1 SV = 1 | 3.6 |
| RN220_BOVIN | E3 ubiquitin-protein ligase RNF220 OS = Bos taurus GN = RNF220 PE = 2 | |
| | SV = 1 | 3.6 |
| FACR2_BOVIN | Fatty acyl-CoA reductase 2 OS = Bos taurus GN = FAR2 PE = 2 SV = 1 | 6.7 |
| GBRR2_BOVIN | Gamma-aminobutyric acid receptor subunit rho-2 OS = Bos taurus | |
| | GN = GABRR2 PE = 2 | |
| | SV = 4 | 5.5 |
| IL15_BUBBU | Interleukin-15 OS = Bubalus bubalis GN = IL15 PE = 2 SV = 1 | 5.6 |
| IL2_CEREL | Interleukin-2 OS = Cervus elaphus GN = IL2 PE = 2 SV = 1 | 5.6 |
| IL4_BUBBU | Interleukin-4 OS = Bubalus bubalis GN = IL4 PE = 2 SV = 1 | 5.6 |
| K1C10_BOVIN | Keratin, type I cytoskeletal 10 OS = Bos taurus GN = KRT10 PE = 3 SV = 1 | 5.6 |
| K1C14_BOVIN | Keratin, type I cytoskeletal 14 (Fragment) OS = Bos taurus GN = KRT14 | |
| | PE = 2 SV = 1 | 5.5 |
| K1C15_SHEEP | Keratin, type I cytoskeletal 15 OS = Ovis aries GN = KRT15 PE = 2 SV = 1 | 5.6 |
| K1C17_BOVIN | Keratin, type I cytoskeletal 17 OS = Bos taurus GN = KRT17 PE = 2 SV = 1 | 5.5 |
| KT222_BOVIN | Keratin-like protein KRT222 OS = Bos taurus GN = KRT222 PE = 2 SV = 1 | 5.5 |
| KIF22_BOVIN | Kinesin-like protein KIF22 OS = Bos taurus GN = KIF22 PE = 2 SV = 2 | 3.5 |
| PERL_BOVIN | Lactoperoxidase OS = Bos taurus GN = LPO PE = 1 SV = 1 | 3.3 |
| TRFL_BUBBU | Lactotransferrin OS = Bubalus bubalis GN = LTF PE = 1 SV = 1 | 3.3 |
| MAP2_BOVIN | Methionine aminopeptidase 2 OS = Bos taurus GN = METAP2 PE = 2 | |
| | SV = 1 | 2.2 |
| MYLK_SHEEP | Myosin light chain kinase, smooth muscle (Fragment) OS = Ovis aries | |
| | GN = MYLK PE = 2 S | 2.1 |
| PARP1_BOVIN | Poly [ADP-ribose] polymerase 1 OS = Bos taurus GN = PARP1 PE = 2 | |
| | SV = 2 | 5.4 |
| RSRC1_BOVIN | Serine/Arginine-related protein 53 OS = Bos taurus GN = RSRC1 PE = 2 | 2.9 |
| | SV = 1 | |
| NAC1_FELCA | Sodium/calcium exchanger 1 OS = Felis catus GN = SLC8A1 PE = 2 SV = 1 | 5.4 |
| SPICE_BOVIN | Spindle and centriole-associated protein 1 OS = Bos taurus GN = SPICE1 | |
| | PE = 2 SV = 1 | 5.6 |
| SMC3_BOVIN | Structural maintenance of chromosomes protein 3 OS = Bos taurus | |
| | GN = SMC3 PE = 1 SV = 1 | 5.3 |
| STX17_BOVIN | Syntaxin-17 OS = Bos taurus GN = STX17 PE = 2 SV = 1 | 2.2 |
| TFR1_HORSE | Transferrin receptor protein 1 OS = Equus caballus GN = TFRC PE = 2 | |
| | SV = 1 | 3.6 |
| TAGL_BOVIN | Transgelin OS = Bos taurus GN = TAGLN PE = 1 SV = 4 | 3.5 |
| ZNHI1_BOVIN | Zinc finger HIT domain-containing protein 1 OS = Bos taurus | |
| | GN = ZNHIT1 PE = 2 SV = 1 | 3.5 |
| ZN184_BOVIN | Zinc finger protein 184 OS = Bos taurus GN = ZNF184 PE = 2 SV = 1 | 3.5 |

### EXAMPLE 9

### Redosing using UAGT in Mice

FIG. 9 illustrates the effect of multiple UAGT treatments on reporter gene expression in the submandibular gland in mice. Ten animals (mice) were used in the study. The treatments in the mice were delivered seven days apart, for a total duration of 21 days. An anti-inflammatory agent was used on the duct opening to allow multiple treatments.

UAGT of Luciferase-expressing plasmid, pCMV-GL3, to the mouse submandibular gland was carried out as previously described in Passineau et al., 2010. Bioluminescent imaging of the submandibular gland was carried out 48 hours after each treatment, for a total of three treatments, spaced one week apart, and three imaging sessions, spaced one week apart. The results are shown below in Table 3.

TABLE 3

| 3 minute exposure | Treatment 1 | Treatment 2 | Treatment 3 |
|---|---|---|---|
| 1585 | 363100 | 15180 | 23510 |
| 1586 | 16940 | 50170 | 99870 |
| 1587 | 18800 | 5557000 | 176100 |
| 1588 | 13060 | 569900 | 10620 |
| 1589 | 140400 | 1353000 | 7177000 |
| 1590 | 62630 | 21210 | 32040000 |
| 1591 | 17140 | 210200 | 3209000 |
| 1592 | 62630 | 572300 | 1855000 |
| 1597 | 18760 | 956700 | 2381000 |
| 1598 | 878500 | 84000 | 137000 |
| Average | 159196 | 938966 | 4710910 |

The results are also shown in FIG. 9. It is noted that the y-axis is a log scale; the straight line slope on this log scale demonstrates synergy between the effects of multiple doses.

The results demonstrate the synergistic effects of serial dosing using UAGT for transfer of a non-viral vector. Similar methods may be used to titrate gene therapy for human treatment, enabling the development and use of dosing regimens tailored to the needs of specific patients.

### EXAMPLE 10

A method employing the assessment of an empirically quantifiable level of xerostomia is used to titrate the number of doses required to achieve the correct dosage level that treats xerostomia in an individual patient presenting with xerostomia.

Xerostomia is evaluated by a Visual Analogue Scale (VAS) based upon the patient's subjective experience of dry mouth.

A patient experiencing xerostomia at an unacceptable level receives one UAGT treatment at a low dosage level (0.1 mg/ml).

One week later, the patient is evaluated again, based upon VAS criteria.

If patient remains in an unacceptable clinical state, the patient is given another treatment.

Seven days later, follow up evaluation of the patient is performed using VAS criteria.

By repeating the steps involving UAGT treatment and evaluation of xerostomia, the total number of treatments and total dosage of expression vector are titrated to the individual patient's condition.

It is to be understood that this disclosure is not limited to the particular processes, compositions, or methodologies described, as these may vary. It is also to be understood that the terminology used in the description is for the purpose of describing some embodiments, and is not intended to limit the scope of the present disclosure.

Where features or aspects of the disclosure are described in terms of a Markush group or other grouping of alternatives, those skilled in the art will recognized that the disclosure is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the disclosure.

All references cited herein are incorporated by reference herein in their entireties.

What is claimed is:

1. A method for gene transfer into a salivary gland of a subject comprising:
contacting a salivary gland of the subject with a composition comprising microbubbles and a non-viral expression vector comprising a nucleic acid sequence encoding a water channel, wherein the non-viral vector is present in the composition in an amount from about 0.01 μg/μl to about 1 μg/μl; and
applying ultrasound to the salivary gland.

2. The method of claim 1 wherein the salivary gland is a parotid gland.

3. The method of claim 1 wherein the salivary gland is a submandibular gland.

4. The method of claim 1 wherein the water channel is aquaporin-1.

5. The method of claim 1 wherein the non-viral vector is present in the composition in an amount of from about 0.1 μg/μl to about 1 μg/μl.

6. The method of claim 1 wherein the non-viral vector is present in the composition in an amount of from about 0.01 μg/μl to about 0.1 μg/μl.

7. The method of claim 1 wherein saliva volume is increased in the contacted salivary gland.

8. The method of claim 2 wherein saliva volume is increased in an untreated contralateral salivary gland.

9. A method for treating xerostomia and/or hyposalivation in a subject in need of treatment comprising:
contacting a salivary gland of the subject with a composition comprising microbubbles and a non-viral expression vector comprising a nucleic acid sequence encoding a water channel, wherein the non-viral vector is present in the composition in an amount from about 0.01 μg/μl to about 1 μg/μl; and
applying ultrasound to the salivary gland.

10. The method of claim 9 wherein the salivary gland is a parotid gland.

11. The method of claim 9 wherein the salivary gland is a submandibular gland.

12. The method of claim 9 wherein the water channel is aquaporin-1.

13. The method of claim 9 wherein the non-viral vector is present in the composition in an amount of from about 0.1 μg/μl to about 1 μg/μl.

14. The method of claim 9 wherein the non-viral vector is present in the composition in an amount of from about 0.01 μg/μl to about 0.1 μg/μl.

15. The method of claim 9 wherein saliva volume is increased in the treated salivary gland.

16. The method of claim 10 wherein saliva volume is increased in an untreated contralateral salivary gland.

17. A method of increasing salivary flow from an undamaged salivary gland in a subject comprising:
contacting a salivary gland of the subject with a composition comprising microbubbles and a non-viral expression vector comprising a nucleic acid sequence encoding a water channel, wherein the non-viral vector is present in the composition in an amount from about 0.01 μg/μl to about 1 μg/μl; and
applying ultrasound to the contacted salivary gland,
wherein salivary flow is increased from the undamaged contralateral salivary gland.

18. The method of claim 17 wherein the salivary gland is a parotid gland.

19. A method of treatment for xerostomia comprising:
A) providing to a salivary gland of a subject in need thereof a treatment comprising administering a composition comprising microbubbles and a non-viral expression vector comprising a nucleic acid sequence encoding a water channel, and applying ultrasound to the salivary gland;
B) assessing the level of xerostomia in the subject using a visual analog scale (VAS);
C) if the assessment indicates that xerostomia remains present in the subject, providing further treatment to the salivary gland, wherein the treatment comprises administering a composition comprising microbubbles and a non-viral expression vector comprising a nucleic acid sequence encoding a water channel, and applying ultrasound to the salivary gland; and
D) optionally, repeating the above steps A-C until assessment by visual analog scale indicates that xerostomia has been relieved in the subject.

20. The method of claim 19 wherein the non-viral vector is present in the composition in an amount of from about 0.01 μg/μl to about 0.2 μg/μl.

21. The method of claim 19 wherein the level of xerostomia is assessed in step B at about 1 week after the treatment according to step A.

22. The method of claim 19 wherein the level of xerostomia is assessed in step B at about 1 week to about 2 weeks after the treatment according to step A.
